(19) United States
(12) Patent Application Publication
(10) Pub. No.: US 2014/0205650 A1
(43) Pub. Date: Jul. 24, 2014

(54) COATED SUPPOSITORIES

(71) Applicant: Thorsteinn Loftsson, Reykjavik (IS)

(72) Inventor: Thorsteinn Loftsson, Reykjavik (IS)

(73) Assignee: LIPID PHARMACEUTICALS EHF., Reykjavik (IS)

(21) Appl. No.: 14/342,931

(22) PCT Filed: Sep. 6, 2012
(86) PCT No.: PCT/IS2012/050012

§ 371 Date: Mar. 5, 2014

(30) Foreign Application Priority Data
Sep. 6, 2011 (IS) 8980

(51) Int. Cl. A61K 9/02; A61K 31/20; A61K 31/573; A61K 35/60; A61K 31/167; A61K 31/202; A61K 31/201

(52) U.S. Cl. CPC A61K 9/025; A61K 31/202; A61K 31/20; A61K 31/201; A61K 35/60; A61K 31/167; A61K 31/573
USPC 424/433; 514/560; 514/171; 424/422; 427/2.14

(57) ABSTRACT

The invention relates to coating of suppositories containing free unsaturated fatty acid, fatty acid ethyl ester and fatty acid monoglyceride that are susceptible to oxidative degradation. The coating retards oxidative degradation of unsaturated fatty acids and gives the suppository a non-oily and smooth surface. The coating will enhance the shelf-life of the suppository and allow their storage at room temperature for extended time period. The coating may or may not contain medicament such as local anesthetic or steroid. According to this invention chemically unstable fatty acids in coated suppositories can be used to stimulate the process of defecation or to treat disorders such as hemorrhoids, bacterial infections, viral infections and inflammations, as well as against fissura ani and pruritus ani. Furthermore, coating suppositories, that contain high concentrations of free fatty acids, can reduce acid induced rectal irritation.

### TECHNICAL BACKGROUND AND PRIOR ART

Rectal suppositories are solid dosage forms that are inserted into the rectum, most often with the fingers. After insertion, suppositories soften and melt, disperse or dissolve in the cavity fluids. Rectal suppositories for adults are usually about 2 grams and those for infants about half that weight. Rectal suppositories are used for both systemic and local drug delivery. Suppositories should be stable and solid at temperatures below about 30° C. but once inserted should soften and melt quickly distributing its active ingredients to the surrounding tissue (Block, 2006). Low-melting soft suppositories should be stored in a refrigerator. Rectal suppositories intended for local action are used to relieve constipation and inflammation, pain and itching associated with hemorrhoids, as well as other medical conditions of the anorectal area, including bacterial and viral infections. Suppositories are most commonly prepared by molding but they can also be prepared by compression and rolling.

Suppositories can be protected by film coating (Gilbert, 1966) or other types of protective coating (Gross, 1953; Fessenden, 1949) although this is not commonly used in commercial products. Rectal suppositories can also be characterized as rectal capsules where a solid, semi-solid or oil core is in, for example, a gelatin or cellulose ether capsule (Bauer, 1969; Takagishi, 1983). Dual-layer suppositories where, for example, local anesthetic is located in a rapid release outer layer and a healing emollient, such as cod-liver oil, in the inner core have also been described (Hetterick, 1954; Sperti, 1968).

Laxatives are used to treat constipation, i.e. the absence of regular defecation, accumulation of feces in the colon and/or the passage of small amounts of hard, dry stools. People who are constipated may find it difficult and painful to have a bowel movement. Laxatives are also used to cleanse the lower bowel before a proctoscopy, rectoscopy, colonoscopy, x-ray imaging of the colon or similar diagnostic procedure. Laxatives may be for oral administration, e.g. tablets, capsules and liquids, or for rectal administration, e.g. suppositories and enemas. Orally administered laxatives can reduce bioavailability of drugs and nutrients.

Castor oil is a well known orally administered laxative with a usual therapeutic adult dose for laxative effect is 15 to 60 mL. About 90% of the fatty acid content in castor oil is the triglyceride formed from ricinoleic acid (12-hydroxy-9-cis-octadecenoic acid), a monounsaturated fatty acid, which is the active component of castor oil, and acts as a laxative by stimulating secretion of fluid and electrolytes in the small intestines. One or two copious of semi-fluid stools are released within 2 to 6 hours of the administration. Ricinoleic acid is effective in preventing the growth of numerous species of viruses, bacteria, yeasts and molds, and it does possess some anti-inflammatory effect (Vieira, 2000; Burdock, 2006).

Lubiprostone (difluoropentyl-2-hydroxy-6-oxooctahydrocyclopenta-heptanoic acid) is a bicyclic fatty acid derived from a metabolite of prostaglandin E1. After oral administration lubiprostone activates specific chloride channels (CIC-2 channels) in the gastro-intestinal tract to stimulate intestinal fluid secretion, increase GI transit, and improve symptoms of constipation (Lacy, 2008). Thus, lubiprostone has a receptor specific effect.

It has been documented that saturated and unsaturated fatty acids possess both antibacterial and antiviral activity, and that the fatty acids play a role in the natural defense against infections in mucosal membranes and skin. In vitro studies have shown that free fatty acids kill enveloped viruses, such as Herpes simples-1 and Herpes simplex-2, Gram-positive bacteria, Gram-negative bacteria, such as Helicobacter pylori, and fungi (Khulushi, 1995; Thormar, 2007; Carballeira, 2008).

Unsaturated fatty acids, such as eicosapentaenoic acid (20:5 n-3, EPA), reduce inflammation and pain (Calder, 2003; Adam, 2003). The dietary and nutritional benefits of essential fatty acids are well known and dietary supplements such as fish oils have been used for a long time, providing polyunsaturated fatty acids (PUFAs), also referred to as highly-unsaturated fatty acids (HUFAs), in the form of triacylglycerides (TAGs) also called triglycerides. The so called essential omega-3 fatty acids are particularly beneficial.

Suppositories containing saturated and unsaturated fatty acids for treating anal disorders such as fissures and ulcers, hemorrhoids, pruritis ani, and other colon-rectal disorders have been described (Kandil, 2003; Kandil, 2005; Kofsky, 2009).

The present inventors have in a pending application proposed to use suppositories with fatty acids as an active ingredient, for treating constipation (Loftsson, 2010).

Free unsaturated fatty acids, and esters and glycerides of unsaturated fatty acid, have a relatively low melting point. When mixed with suppository bases unsaturated fatty acids and their esters give soft suppositories that leave fatty residues on fingers upon administration. Furthermore, rectal administration of suppositories containing unsaturated fatty acids, including polyunsaturated fatty acids, and their esters is hampered by their susceptibility to oxidative degradation and consequent unpleasant odor. Even simple handling of suppositories containing unsaturated fatty acids can give smelly and oily fingers.

Improved fatty acid suppositories with better stability and characteristics for handling would be appreciated.

### SUMMARY OF INVENTION

The invention relates to fatty acid suppositories with protective coating that prevents or retards oxidative degradation of the unsaturated fatty acids and gives them somewhat hard, non-sticky and smooth surface. According to this invention chemically unstable fatty acids can be used in coated suppositories to stimulate the process of defecation or to treat disorders such as hemorrhoids, bacterial infections, viral infections and inflammations, as well as against fissura ani and pruritus ani. Furthermore, the coating according to this invention will substantially enhance the shelf-life of the suppositories and allow their storage at room temperature for extended time periods.

Stability tests of inventors' previously proposed fatty acid suppositories (Loftsson, 2010), after coating according to the present invention, show suprisingly remarkable improvement in stability. Uncoated suppositories containing up to 30 wt % free fatty acids extracted from marine lipids had previously been tested. The color of these relatively soft suppositories turn from light yellow to brown upon storage at room temperature for a few days, unprotected from air but protected from light, releasing a fishy odor.

Coating the suppositories with protective coating that is impermeable or almost impermeable to oxygen prevents or substantially retards oxidative degradation of the unsaturated fatty acids. The coating significantly extends the shelf-life of the suppositories. Furthermore, the coating hardens the relatively soft surface of the suppositories and makes them more convenient to handle. It is an additional effect of the invention that the coating converts soft suppositories, which need to be stored under refrigeration, to hard suppositories that can be stored at room temperature. Further, it is found that preferred suppository coating according to the invention does not affect the bioavailability of the fatty acids.

In a first aspect, the present invention relates to a coated suppository for rectal administration comprising at least one unsaturated fatty acid but preferably a mixture of unsaturated fatty acids that includes polyunsaturated fatty acids. The mixture of unsaturated fatty acids can be derived from but is not limited to vegetable oil, such as corn oil, or marine organisms, such as fish oil. The suppository is coated by a protective coating that retards oxidative degradation of unsaturated fatty acids and gives the suppository a non-oily and smooth surface. The coating will enhance the shelf-life of the suppository and allow their storage at room temperature for extended time period.

A second aspect of the present invention relates to including of biologically active agent, for example, local anesthetic such as lidocaine or anti-inflammatory steroid, such as hydrocortisone, in the coating.

Due to relatively high concentration of unsaturated fatty acids and/or their esters the core will melt at a slightly lower temperature than the coating. Alternatively, a water-soluble coating is dissolved upon administration. The coating can consist of, but not limited to, fatty or oleaginous bases such as cocoa butter, hard fat and hydrogenated vegetable oil, water-soluble or water-miscible bases such as polyethylene glycols, glycol-surfactant combinations and polyoxyethylene sorbitan fatty acid esters, and any combinations thereof. Furthermore, coated suppositories can be in the form of rectal capsules where a free fatty acid-containing core is covered by water-soluble compound such as gelatin.

Although free fatty acids are presently preferred embodiment, salts of free fatty acids with a pharmaceutically acceptable counter ion, fatty acid esters and fatty acid monoglycerides are also within the scope of this invention. Finally, coating suppositories that contain high concentrations of free fatty acids can reduce acid induced rectal irritation.

### DETAILED DESCRIPTION

Unsaturated fatty acids and especially polyunsaturated fatty acids are subjected to autoxidation that occurs under mild conditions in presence of oxygen under formation of peroxides and hydroperoxides. Autoxidation is a free radical chain reaction that can be divided into three stages, i.e. chain initiation, propagation and termination. In the ignition stage free radicals are formed. In the propagation stage the free radicals react with oxygen to form a peroxide radical, which then removes a hydrogen atom from another fatty acid molecule to form a hydroperoxide and in so doing creates a new free radical that continues the cycle. This chain reaction proceeds until oxygen molecules are no longer present or until the free radical molecules are destroyed by, for example, inhibitors and the chain breaks in the termination stage. The unpleasant odor of oxidized fat and oils is due to formation of aldehydes, ketones and short-chain fatty acids, that are degradation products of the hydroperoxides (Pratt, 2011). This type of oxidative degradation is especially common in suppositories containing high concentrations of free unsaturated fatty acids, which are very sensitive to oxidation. This causes the characteristic “fishy” smell of fish oil.

Although free fatty acids are presently a preferred embodiment, salts of free fatty acids with a pharmaceutically acceptable counter ion, fatty acid esters and fatty acid monoglycerides are also within the scope of this invention.

Suppositories are well known in the art. They are generally formulated to be solid at room temperature and up to at least about 30° C. but having a melting temperature below the normal human body temperature of 37° C. It is therefore common to formulate suppositories with a fat base, such as cocoa butter, which fulfils the above melting point criteria. Cocoa butter is a mixture of triglycerides of saturated and unsaturated fatty acids which can be manipulated in solid form at room temperature but melts completely at body temperature. More recent materials include hard fat, macrogols, and various gelatinous mixtures consisting of, for example, gelatin, water and glycerol. Useful commercially available fat bases suitable for the present invention include the above mentioned and in particular Suppocire™ (Gattefossé) lipophilic bases, a semi-synthetic vegetable based oil base available in several grades including Suppocire™ AS, AS2X, NA; Novata™ (Henkel Int.) including Novata A, Novata B, and Novata BC; Witepsol™ (Dynamit Nobel Ab) such as Witepsol™ H5, H12, H15, H32, H35, W25, W31, W32, W32, W35, and W45; Massa Estarinum™ (SASOL), including Massa Estarinum™ of the grades B, BC, E and 2.99; Japocire™ and Ovucire™. The suppositories of the present invention may suitably comprise any of the above mentioned materials as base. Hydrophilic waxes can also be used in the invention, such as the polyethylene glycols (e.g. PEG 1500, PEG 3000, PEG 4000 and mixtures thereof). Suppocire AP, is an amphiphilic base comprising saturated polyglycolyzed glycerides. The suppository dosage form may also in some embodiments comprise further excipients such as but not limited to binders and adhesives, lubricants, disintegrants, colorants and bulking agents. In some embodiments, the suppository comprises a combination of any of the above mentioned base substances.

Conventional coating materials will retard or inactivate rectally administered drugs. The coating methods and coating materials according to this invention will protect the unsaturated fatty acids and make them more convenient to handle without hampering the pharmacological activity of the product.

In embodiments of this invention suppositories, containing one or more fatty acids, are coated by any of the following methods:
coating the inner surface of the suppository mold with the coating material before molding of the suppository,
dipping the suppository in the liquid coating material, —molding or casting the coating material around the suppository,

or coating by some other suitable method.

The coating can comprise, but is not limited to, fatty or oleaginous bases such as cocoa butter, hard fat and hydrogenated vegetable oil, waxes, water-soluble or water-miscible bases such as polyethylene glycols, glycol-surfactant combinations and polyoxyethylene sorbitan fatty acid esters, and combinations thereof. Furthermore, coated suppositories can be in the form of rectal capsules where a free fatty acid containing core is covered by water-soluble compound such as gelatin.

Commercially available materials suitable for coating according to the present invention include in some embodiments Suppocire™ (Gattefossé) lipophilic bases, a semi-synthetic vegetable based oil base available in several grades including but not limited to Suppocire™ AS, AS2X, NA; Novata™ (Henkel Int.) including Novata A, Novata B, and Novata BC; Witepsol™ (Dynamit Nobel Ab) such as Witepsol™ H5, H12, H15, H32, H35, W25, W31, W32, W32, W35, and W45; Massa Estarinum™ (SASOL), incl. Massa Estarinum™ preferably of the grades B, BC, E and 299; Japocire™ and Ovucire™. The coatings of the present invention may suitably comprise any of the above mentioned materials as base. Hydrophilic waxes can also be used in the invention, such as the polyethylene glycols (e.g. PEG 1500, PEG 3000, PEG 4000 and mixtures thereof). Suppocire AP, is an amphiphilic base comprising saturated polyglycolyzed glycerides. Mixtures of the above are as well useful in the invention.

In one embodiment hard fat (e.g. suitable Suppocire grade material) constitutes a substantial fraction of the coating material, such as in the range of about 40-95 wt % of the coat and preferably in the range of 50-85 wt %, such as in the range of 75-90 wt %, mixing other suitable materials with the dominant fraction, such as beeswax, e.g. in the range 5-60 wt %, and preferably in the range 5-40 wt %, such as in the range 5-25 wt % of the coat.

By varying the relative amounts of components in the coat, desired characteristics can be adjusted, such as the softening time and dintiegration time. A higher amount of hydrophobic waxes such as beeswax generally will increase softening times as well as disintegration times, as demonstrated in the accompanying examples.

The coating material preferably also includes a lubricant such as but not limited to glyceryl dibhenate, which may suitably be added in an amount of about 1-5 wt %, such as in the range of 2-3.5 wt % of the coat.

In a useful embodiment of the invention, one or more pharmaceutically active ingredient is added to the coating, such as but not limited to a locally acting anesthetic and/or analgesic such as but not limited to lidocaine, prilocalne, benzocaine, chloroprocaine, bupivacaine, levobupivacaine or other local anesthetic of the amoniester or aminoamide type, or a mixture of such.

Such pharmaceutically active ingredient can be comprised in the coating in a suitable range, depending on the desired dose, such as from 0.1 wt % to 3 wt % of the coat, or sufficient to provide a dose of said agent in the range of about 0.1 mg to about 20 mg.

Other pharmaceutical compounds may be included in the coating, especially when rapid local delivery is desired, such as but not limited to hydrocortisone.

Suppository dosage forms of the invention will generally comprise in the range of 50-2000 mg of the fatty acid active ingredient, and preferably in the range of 50-1000 mg, such as in the range of 100-750 mg, including about 100 mg, about 200 mg, about 300 mg, about 400 mg or about 500 mg. Smaller suppositories for pediatric use are also within the scope of the invention, which generally would be smaller and comprising in the range of 50-750 mg fatty acid active agent, such as in the range of 50-500, e.g. about 50 mg, about 75 mg, about 100 mg, about 200 mg, about 300 mg or about 400 mg. Depending on the desired dose and the desired total size of the suppository the amount of fatty acid active ingredient may comprise in the range of about 5% by weight to about 75% by weight of the total weight of the core, such as in the range of about 5-50% by weight of the core, including in the range of about 10-50% by weight, such as in the range of about 10-40% by weight. These percentage values generally refer if not otherwise noted to the total weight of the core of the suppository but in other embodiments, the percentages can refer to the coated suppositories.

A common size of molded or kneaded suppositories for adult use according to the invention is in the range of about 2-3 mL, such as about 2.0 mL, about 2.2 mL or about 2.5 mL. Depending on the excipient composition, this would generally correspond to a weight of the uncoated core in the range of about 1.5 to about 3 g, accordingly, the suppositories according to the invention are suitably in said weight range, such as having a core of about 1.8 g, about 2.0 g, about 2.2 g or about 2.5 g. A suitable size for pediatric suppositories would generally be about half the above size, such as in the range of 0.5-1.5 mL, e.g. about 0.5 mL, about 0.8 mL, about 1.0 mL, about 1.2 or about 1.5 mL. Common thickness of the coating layer covering the soft suppository core is about 0.1 to 3 mm.

The one or more fatty acid preferably has a chain length in the range of four to 36 carbon atoms, such as a chain length in the range of 4 to 24 and more preferably a chain length in the range of 8 to 24 carbons. More preferably the one or more fatty acid comprise a mixture of fatty acids, which can be derived from suitable natural lipid material such as oils of animal or vegetative origin, fractions thereof or a mixture thereof.

Unsaturated fatty acids useful in the invention include palmitoleic acid (16:1 n-7), cis-vaccenic acid (18:1 n-7), oleic acid (18:1 n-9), elaidic acid (18:1), linoleic acid (18:2 n-6), alpha-linolenic acid (18:3 n-3), gamma-linolenic acid (18:3 n-6), moroctique acid (18:4 n-3), arachidonic acid (20:4 n-6), gadoleic acid (20:1 n-11), gondoic acid (20:1 n-9), cis-11 eicosenoic acid (20:1 n-7), eicosapentaenoic acid (20:5 n-3; EPA), erucic acid (22:1 n-9), cetoleic acid (22:1 n-11), clupanodonic acid (22:5 n-3) and docosahexaenoic acid (22:6 n-3; DHA).

Useful vegetable oils as raw materials for the fatty acids of the invention include safflower oil, corn oil, almond oil, sesame oil, soybean oil, linseed oil, rapeseed oil, grape seed oil, sunflower oil, wheat germ oil, hemp oil, and any mixtures thereof.

In preferred embodiments the fatty acids are derived from oil material which is pharmaceutically acceptable and defined according to Pharmacopoeia standards (pharmaceutical grade oils). Such oils include marine omega oils such as Omega-3 Fish Oil (Lýsi, Iceland).

It has been found useful to include in the suppository dosage form of the invention an excipient oil component such as a triacylglyceride oil, to reduce discomfort during action of the medicament and bowel movements. Accordingly, the dosage form of the invention preferably comprises in the range of about 5-35% by weight of the core triacylglyceride oil, such as more preferably in the range of about 5-25% by weight of the core, such as about 5% by weight, about 10% by weight, about 15% by weight or about 20% by weight. The base is in the embodiments composed accordingly in order to have a desired melting point of the overall composition of the dosage form.

Although the coating will allow production of preservative-free suppositories it can sometimes be useful to include anti-oxidants in the dosage forms of the invention, such as but not limited to butylated hydroxyanisole (BHA), butylated hydroxytoluene (BHT), ascorbic acid or a salt thereof, a sulfatide salt, citric acid, propyl gallate, alpha-tocopherol, and ascorbyl palmitate. Depending on the selected antioxidant compound, a suitable amount is e.g. in the range of about 0.1-0.5% by weight, such as in the range of 0.1-0.3% by weight. Further preservative agents may included in some embodiments, such as any of those of the group consisting of benzoic acid or derivatives thereof, including of C₁₋₆-alkyl-p-hydroxy-benzoic acids, such as methyl-p-hydroxy-benzoic acid, ethyl-p-hydroxy-benzoic acid, propyl-p-hydroxy-benzoic acid, butyl-p-hydroxy-benzoic acid, and mixtures thereof. In a particular interesting embodiment, the preservative is a mixture of methyl-p-hydroxy-benzoic acid and propyl-p-hydroxy-benzoic acid, in the proportion of from about 3:1 to about 5:1 by weight, preferably in the proportion of about 4:1 by weight. The preservative or preservatives is/are preferably present in the formulation in such a concentration of about 0.05-0.2% by weight calculated on the formulation, that it does not to any substantial extent impair the activity of the lipid or lipids.

In another aspect, the invention provides a method for stimulating and/or initiating the process of defecation, which comprises administering coated rectal suppositories containing one or more fatty acids. The method is based on the stimulating effect of the fatty acids on the polymodal nocireceptors in the rectal mucosa. The fatty acid is preferably selected from any of the above mentioned fatty acids and mixtures of fatty acids and can be formulated in a suitable form such as in any of the forms described above.

Still another aspect of this invention provides a method for use of fatty acids to treat disorders such as hemorrhoids, bacterial infections, viral infections (e.g. herpes simplex virus infections) and inflammations, as well as against fissura ani and pruritus ani, which comprises administering coated rectal suppositories containing one or more fatty acids.

As can be understood from the above discussion, free fatty acids are the preferred form of fatty acids in the method, although other forms are contemplated, such as fatty acid ethyl esters, salts of fatty acids and fatty acid monoglycerides.

The method will generally comprise administering in the range of about 100 to 2000 mg fatty acids, such as in the range of 100-1000 mg, or any of the above mentioned ranges and amounts, in coated suppositories.

Fatty acids for use in the invention can be suitably provided by hydrolysis of natural oils such as those above mentioned. Hydrolysis of triglycerides can be acid or base catalyzed. In the below Example 1 is described how a preferred extract of free fatty acids is produced by acid hydrolysis of a marine fish oil. Accordingly, a fatty acid mixture obtainable from hydrolysis of natural oil, such as from a vegetable oil or fish oil, for sue as a laxative, anti-inflammatory, antibacterial and antiviral medicament is included in the invention. The fatty acid of the invention is formulated as a coated suppository, preferably as further described herein.

Previously we have manufactured and tested uncoated suppositories containing up to 30% wt free fatty acids extracted from marine lipids (Loftsson, 2010). Such suppositories can conveniently be coated according to the present invention. In a preferred embodiment the one or more fatty acids comprise a mixture of fatty acids comprising at least about 20% by weight of unsaturated fatty acids and thereof at least about 5% by weight polyunsaturated fatty acids or PUFA. The term poly-unsaturated fatty acid indicates a fatty acid with more than one double bond in its acyl side chain and is used herein interchangeable with the term highly-unsaturated fatty acid or HUFA. Many natural oils provide such fatty acid composition, e.g. the vegetable oils mentioned above, and fish oils and other marine oils provide a high fraction of PUFA. Among poly-unsaturated fatty acids are the omega-3 fatty acids alpha-linolenic acid (18:3 n-3), stearidonic acid (18:3), moroctic acid (18:4 n-3), eicosatrienoic acid (20:3), eicosatetraenoic acid (20:4), eicosapentaenoic acid (20:5 n-3; EPA), docosapentaenoic acid (22:5 n-3; clupanodonic acid), and docosahexaenoic acid (22:6 n-3; DHA), tetracosapentaenoic acid (24:5), and tetracosahexaenoic acid (24:6). Other useful polyunsaturated fatty acids are omega-6 fatty acids including linoleic acid (18:2 n-6), gamma-linolenic acid (18:3 n-6), eicosadienoic acid (20:2 n-6) and docosapentaenoic acid (22:5 n-6; osbond acid). The designation in parentheses indicates the total number of carbon atoms in the acyl chain and the number of double bonds, thus 18:3 is a fatty acid with 18 carbon atoms and three double bonds. The omega number indicates how far from the lipophilic end of the acyl chain the first double bond is situated, also indicated with n, as is used for other unsaturated fatty acids above.

In a useful embodiment, the pharmaceutical dosage form comprises a mixture of fatty acids derived from marine organisms. Marine organisms useful as sources of the fatty acid material include marine animal oil derived from an animal source selected from fish liver oil including cod liver oil, tuna oil; fish flesh or fish meal including flesh or meal from herring, capelin, mackerel, menhaden, sardine, anchovy, horse mackerel, blue whiting, and tuna; planktonic organisms, squid and molluscs.

Ethyl esters of fatty acids for use in the invention can be obtained by esterification of free fatty acids such as with a suitable lipase, such as but not limited to lipase from Rhizomucor miehei (MML), Pseudomonas sp. Lipase (PSL) and Pseudomonas fluorescens lipase (PFL). See e.g. Halldorsson et al (2004), Breivik 1995WO95/24459, WO 00/49117 and U.S. Pat. No. 7,491,522.

Monoglycerides can be obtained by selective esterification with glycerol with lipase under suitable reaction conditions, for an overview see Osman et at (2006).

Suppository any of claims 1-11, wherein said coating comprises a substance selected from a semi-synthetic vegetable based oil base such as Suppocire™ (Gattefosse), Novata™ (Henkel Int.), Witepsol™ (Dynamit Nobel Ab), Massa Estarinum™ (SASOL), hydropylic waxes such as polyethylene glycols, water-insoluble waxes such as beeswax, or mixtures thereof.

### EXAMPLES

### Example 1

### Preparation of Fatty Acid Extract

Preparation of a fatty acid mixture from fish oil: The fatty acid mixture is extracted from fish oil (such as fish-liver oil, for example cod-liver oil) after hydrolysis in aqueous media. Sodium hydroxide (130 g) is dissolved in a mixture of 1.0 L of ethanol and 1.5 L of purified water. Then 1000 g of cod-liver oil is added and the mixture heated under reflux at 85° C. for 8 hours. Then after cooling to 5° C. 800 ml of 6M hydrochloric acid is added and the oil phase separated from the aqueous solution. The oil is then washed four times with 800 ml of purified water at 50° C. and finally dried at room temperature under vacuum. The fatty acid composition of the extract and the cod-liver oil used to prepare the extract is determined by gas-chromatography. The relative fatty acid composition of the extract is approximately the same as in the unhydrolyzed oil (Table 1).

TABLE 1
The fatty acid composition of triglycerides found in cod-liver oil and its
fatty acid extract.

| | | Composition (%) | |
|---|---|---|---|
| Fatty acid | | Cod-liver | |
| Name | Number | oil | Fatty acid extract |
| Myristic acid | 14:0 | 3.4 | 3.8 |
| Palmitic acid | 16:0 | 10.2 | 11.4 |
| Palmitoleic acid | 16:1 n-7 | 6.6 | 7.0 |
| Stearic acid | 18:0 | 2.3 | 2.5 |
| cis-Vaccenic acid | 18:1 n-7 | 4.4 | 4.4 |
| Oleic acid | 18:1 n-9* | 17.6 | 18.8 |
| Linoleic acid | 18:2 n-6 | 1.2 | 1.3 |
| Moroctique acid | 18:4 n-3 | 2.1 | 2.1 |
| cis-11-Eicosenoic acid | 20:1 n-7 | 0.4 | 0.5 |
| Gondoic acid | 20:1 n-9 | 9.6 | 9.4 |
| Gadoleic acid | 20:1 n-11 | 1.9 | 2.1 |
| Eicosapentaenoic acid (EPA) | 20:5 n-3 | 8.3 | 7.5 |
| Erucic acid | 22:1 n-9 | 0.6 | 0.6 |
| Cetoleic acid | 22:1 n-11 | 9.0 | 9.7 |
| Clupandonic acid | 22:5 n-3 | 1.4 | 1.4 |
| Docosahexaenoic acid (DHA) | 22:6 n-3 | 11.1 | 9.7 |

*includes linolenic acid (18:3 n-3) that was not separated from 18:1 n-9 in the GC system. Cod-liver oil usually contains less than 1% linolenic acid.

### Example 2

### Uncoated Suppositories with Fatty Acid Extract

Uncoated suppositories were prepared by the molding (fusion) method. Beeswax (Apifil Gattefossé, France; 50 g), glycerol dibehenate (Compritol 888, Gattefossé; 20 g) and hard fat (Suppocire NA 0, Gattefossé; 530 g) were melted and mixed at about 75° C. and allowed to cool to 50° C. Then cod-liver oil (100 g) and the fatty acid extract (300 g) were added and after thorough mixing and cooling to 45° C. the mixture was poured into a suppository mold (2.2 ml) and cooled at room temperature.

### Example 3

### Coating of Suppositories with Hard Fat by a Dipping Method

Hard fat (Suppocire NA 0, Gattefossé) was melted in a beaker and cooled to about 40° C. Suppositories attached to a tiny string were prepared according to Example 2 and allowed to cool in a refrigerator. Then the cool suppositories were dipped for couple of seconds into the melted hard fat and quickly removed from the beaker and allowed to cool at room temperature. This was repeated couple of times until hard fat coating of desired thickness was obtained.

Alternatively the suppositories were coated by dipping them into melted polyethylene glycol 1000.

### Example 4

### Coating of Suppositories with Hard Fat by Mold-Coating

Hard fat (Suppocire NA 0, Gattefossé) was melted in a beaker and allowed to cool to about 60° C. The melted hard fat was poured into a suppository mold (2.2 ml) and again drained from the mold. This was repeated until the inner surface of the mold was evenly covered by had fat coating of desired thickness. Then the suppository was molded as described in Example 2.

### Example 5

### Coating of Suppositories Double-Molding

Uncoated suppositories were prepared by the molding (fusion) method. Beeswax (Apifil Gattefossé, France; 50 g), glycerol dibehenate (Compritol 888, Gattefossé; 20 g) and hard fat (Suppocire NA 0, Gattefossé; 530 g) were melted and mixed at about 75° C. and allowed to cool to 50° C. Then cod-liver oil (100 g) and the fatty acid extract (300 g) were added and after thorough mixing and cooling to 45° C. the mixture was poured into a suppository mold (1.15 ml) and cooled in a refrigerator. Hard fat was melted, poured into a suppository mold (2.2 ml), allowed to cool to about 45° C. and the smaller (1.15 ml) suppository placed in the center of this larger one. Then the double molded suppository was allowed to cool at room temperature.

### Example 6

### The Coating Composition

Several different mixtures of hard fat (Suppocire NA 0, Gattefossé) and beeswax (Apifil, Gattefossé), weight ratio 40:60 to 85:15 (Suppocire:Apifil), were melted in a beaker and allowed to cool to about 60° C. The melted mixture was poured into a suppository mold (2.2 ml) and again drained from the mold. This was repeated until the inner surface of the mold was evenly covered by coating of desired thickness. Then the suppository was molded as described in Example 2.

The softening time of the suppositories, as well as suppositories from Example 4, was determined according to the method of the European Pharmacopoeia 7.0 (2.9.22 Softening time determination of lipophilic suppositories, p. 288, 2011), Apparatus A. The disintegration of the suppositories, as well as suppositories from Example 4, was determined according to the method of the European Pharmacopoeia 7.0 (2.9.2 Disintegration of suppositories and pessaries, p. 255-256, 2011). The time indicated is the time from the suppository was placed in the liquid medium until it had melted and the oil collected on the surface of the liquid. See Table 2.

TABLE 2
The effect of hard fat:beeswax weight ration of the coating on the
physical properties of the suppositories.

| Hard fat:beeswax | Average coat | Softening time | Disintegration |
|---|---|---|---|
| weight ration | thickness (mm) | (minutes) | test (minutes) |
| 40:60 | 2.4 | >6 | >30 |
| 50:50 | 2.4 | 5.5 | >30 |
| 60:40 | 1.7 | 4.6 | >30 |
| 70:30 | 1.3 | 5.0 | >30 |
| 75:25 | 1.7 | 5.3 | 10 |
| 80:20 | 2.0 | 4.5 | 10 |
| 85:15 | 1.0 | 4.5 | 10 |
| 100:0 | 1.2 | 4.0 | 10 |
| No coating | — | 2.9 | 10 |

### Example 7

### Medicated Coating

Mixture of hard fat (Suppocire NA 0, Gattefossé) and beeswax (Apifil, Gattefossé), weight ratio 85:15 (Suppocire:Apifil), were melted in a beaker and allowed to cool to about 60° C. Appropriate amount of lidocaine or hydrocortisone was suspended in the hot coating mixture containing either 5 wt % lidocaine or 1 wt % hydrocortisone. The mixture was poured into a suppository mold (2.2 ml) and again drained from the mold. This was repeated until the inner surface of the mold was evenly covered by coating of desired thickness. Then the suppository was molded as described in Example 2.

The softening time of the suppositories was determined according to the method of the European Pharmacopoeia 7.0 (2.9.22 Softening time determination of lipophilic suppositories, p. 288, 2011), Apparatus A. The disintegration of the suppositories was determined according to the method of the European Pharmacopoeia 7.0 (2.9.2 Disintegration of suppositories and pessaries, p. 255-256, 2011). The time indicated is the time from the suppository was placed in the liquid medium until it had melted and the oil collected on the surface of the liquid. See Table 3.

TABLE 3
The effect of addition of lidocaine free base and hydrocortisone to
the hard fat:beeswax (weight ratio 85:15) coating on the physical
properties of the suppositories.

| | Average coat | Softening time | Disintegration |
|---|---|---|---|
| Coating | thickness (mm) | (minutes) | test (minutes) |
| 5 wt % lidocaine | 2.7 | 4.7 | 10 |
| 1 wt % hydrocortisone | 1.3 | 6.3 | 10 |

### Example 8

### Availability Comparison—Coated Suppositories Vs. Uncoated Suppositories

To estimate the bioavailability of the active ingredients five healthy volunteers participated in this study. On day one they administered rectally one uncoated suppository prepared in Example 2 and on day seven they administered an identical but coated suppository having a hard fat:beeswax (weight ratio 85:15) coating and prepared according to Example 6. Both the uncoated and the coated suppositories containing the fish-liver oil and the fatty acid mixture stimulated bowel movement causing defecation in all participants. The study shows that the coating did not affect the bioavailability of the active ingredients, i.e. the bioavailability of the fatty acid mixture.

### Example 9

### Shelf-Life Comparison—Coated Suppositories Vs. Uncoated Suppositories

Ten uncoated suppositories produced according to Example 2 and ten coated suppositories prepared according to Example 6 and coated with hard fat:beeswax (weight ratio 85:15) were placed in open containers and stored at room temperature (23° C.). Before storage the suppositories were removed from the molds. The stability of the suppositories was judged by evaluating their color, smell and surface. The freshly prepared suppositories, both the coated and the uncoated, were pale yellow but the uncoated suppositories gradually became more yellow to brown during storage (Table 4). Also, the uncoated suppositories soon started to release fishy smell that gradually became stronger during storage. After a few days the uncoated suppositories got an oily surface and stuck to the bottom of the container while the coated ones had a hard surface and did not stick to the container.

TABLE 4
The effect coating on the oxidative degradation of free unsaturated fatty acids in
suppositories. Effect of storage in an open container at room temperature (23° C.).

| | Average coat | One week | | Two weeks | | Fifty two weeks | |
|---|---|---|---|---|---|---|---|
| Suppository | thickness (mm) | Color | Smell | Color | Smell | Color | Smell |
| Uncoated | — | Pale | Fish | Dark | Strong | Brown | Strong |
| | | yellow | odor | yellow | fish | | fish |
| | | | | | odor | | odor |
| Coated | 1.0 | Almost | No | Almost | No | Almost | No |
| | | white | odor | white | odor | white | odor |

The example demonstrates surprisingly increased stability of the suppositories by coating

### REFERENCES

Adam O.: Dietary fatty acids and immune reactions in synovial tissue, Eur. J. Med. Res., 8, 381-387, 2003.
Banker, G. S.: Film coating theory and practice, J. Pharm. Sci., 55, 81-89, 1966.
Bauer K., Coatings for rectal capsules and process for their manufacturing. U.S. Pat. No. 3,432,594, 11 Mar. 1969.
Block, L. H.: Medicated topical. In Remington, the science and practice of pharmacy, 21^{st} Edition, Lippincott Williams & Wilkins, Philadelphia, 2006, Chapter 44, pp. 871-888.
Breivik H., Haraldsson, G. G., Intl. patent application No. WO 95/24459 (14 Sep. 1995).
Burdock G. A., et al., Toxicology and pharmacology of sodium ricinoleate, Food Chem. Tox., 44, 1689-1698, 2006.
Calder P. C.: Long-chain n-3 fatty acids and inflammation: potential application in surgical and trauma patients, Braz. J. Med. Biol. Res., 36, 433-446, 2003.
Carballeira N. M., New advances in fatty acids as antimalarial, antimycobacterial and antifungal agents, Prog. Lipid Res., 47, 50-61, 2008).
Fessenden G. R.: Polyvinyl-alcohol-coated suppository, U.S. Pat. No. 2,477,292; 1949.
Gross H. M., Becker C. H.: A study of suppository Bases. I. Review of Literature, J. Am. Pharm. Assoc., 42, 90-95, 1953.
Haraldsson, G. G., et al., Intl. patent application No. WO 2004/043894 (27 May 2004).
Haraldsson, G. G., Thorstad O., Intl. patent application No. WO 00/49117 (24 Aug. 2000).
Hetterick R. D.: Dual element suppository, U.S. Pat. No. 2,696,456, 7 Dec. 1954.
Kandil O., Polyunsaturated fatty acid fractions of Nigella sativa L. seeds. US Patent Appl. No. 2003/0060508, Mar. 27, 2003.
Kandil O., Polyunsaturated fatty acid fractions of Nigella sativa L. seeds. US Patent Appl. No. 2005/0214241, Sep. 29, 2005.
Khulushi, S., et al. The effect of unsaturated fatty acids on Helicobacter pylori in vitro, J. Med. Microbiol., 42, 276-282, 1995.
Kofsky: Omega-3 essential fatty acid composition and method for treatment of anal and colon-rectal disorders. U.S. patent application Ser. No. 12/414,414, Pub. No.: US 2009/0247494 A1; Oct. 1, 2009.
Lacy B. E., Levy L. C., Lubiprostone: a novel treatment for chronic constipation, Clin. Interv. Aging, 3, 357-364, 2008.
Loftsson T., Stefansson E., Fatty acids for use as a medicament. PCT Application No. WO 2010/049954, May 6, 2010.
Pratt D. A. et al.: Free radical oxidation of polyunsaturated lipids: new mechanistic insights and the development of peroxyl radical clocks, Acc. Chem. Res. 44, 458-467, 2011.
Sperti G. S.: Suppository, U.S. Pat. No. 3,415,249, 10 Dec. 1968.
Takagishi Y.: Medicament capsules for rectal application. U.S. Pat. No. 4,405,597, 20 Sep. 1983.
Thormar H., Hilmarsson H., The role of microbicidal lipids in host defense against pathogens and their potential as therapeutic agents, Chem. Phys. Lip., 150, 1-11, 2007.
Vieira C., et al., Effect of ricinoleic acid in acute and subcronic experimental models of inflammation, Med. Inflammation, 9, 223-228, 2000.

1. A pharmaceutical suppository unit dosage form for rectal, vaginal or urethral administration comprising a core comprising as an active ingredient one or more fatty acids, and a coating enclosing said core.

2. The suppository unit dosage form of claim 1, wherein said one or more fatty acid is in the form selected from free fatty acid, fatty acid ethyl ester and fatty acid monoglyceride.

3. The suppository unit dosage form of claim 1, wherein said one or more fatty acids comprise one or more saturated or unsaturated fatty acid with a carbon chain length in the range of C4 to C36.

4. The suppository unit dosage form of claim 1, wherein said one or more fatty acids comprise a mixture of fatty acids comprising at least about 20% by weight of said core of unsaturated fatty acids of which at least about 5% by weight of said core are polyunsaturated fatty acids.

5. The suppository unit dosage form of claim 4, wherein said mixture of fatty acids comprises fatty acids derived from marine organisms.

6. The suppository unit dosage form of claim 5, wherein said mixture of fatty acids is derived from marine organism material selected from the group consisting of marine animal oil derived from an animal source selected from fish liver oil including cod liver oil, tuna oil; fish flesh or fish meal including flesh or meal from herring, capelin, mackerel, menhaden, sardine, anchovy, horse mackerel, blue whiting, and tuna; planktonic organisms, squid and molluscs.

7. The suppository unit dosage form of claim 1, comprising in the range of 100-2000 mg fatty acids.

8. The suppository unit dosage form of claim 1, comprising in the range of about 5% by weight to 75% by weight of said core fatty acid.

9. The suppository unit dosage form of claim 1, which comprises in the range of 10-50% by weight of said core fatty acids and further comprises thereof in the range of 5-25% by weight triacylglyceride oil.

10. The suppository unit dosage form of claim 1, comprising in the range of about 5 to 75% by weight of said core fatty acids as active ingredient, for stimulating and/or initiating bowel movements, treatment of hemorrhoids, bacterial infections, viral infections including herpes simplex virus infections, and inflammations, as well as against fissura ani and pruritus ani.

11. The suppository unit dosage form of claim 5, comprising a fatty acid mixture derived from fish oil by hydrolyzing the fish oil glycerides to free fatty acids.

12. The suppository unit dosage form according to claim 1, wherein said coating melts below 37° C. and comprises a substance selected from the group consisting of a semi-synthetic vegetable based oil base such as Suppocire™ (Gattefosse), Novata™ (Henkel Int.), Witepsol™ (Dynamit Nobel Ab), Massa Estarinum™ (SASOL), fatty or oleaginous bases such as cocoa butter, hard fat and hydrogenated vegetable oil, waxes, water-soluble or water-miscible bases such as polyethylene glycols, glycol-surfactant combinations and polyoxyethylene sorbitan fatty acid esters, water-insoluble waxes such as beeswax, and any mixtures thereof.

13. The suppository unit dosage form according to claim 12, having a coating comprising in the range from about 5 wt % to about 60 wt % beeswax.

14. The suppository unit dosage form according to claim 12, comprising in the range from about 40 wt % to about 95 wt % hard fat.

15. The suppository unit dosage form according to claim 1, comprising a coating with a thickness in the range from about 1.0 mm to about 2.5 mm.

16. The suppository unit dosage form according to claim 1, wherein said suppository consists of a capsule where a solid, semi-solid or oil core is encapsulated by water-soluble coating such as gelatin or cellulose ether.

17. The suppository unit dosage form according to claim 1, wherein said coating is applied by a method selected from coating the inner surface of the suppository mold with the coating material before molding of the suppository, by dipping the suppository in the liquid coating material, by molding or casting the coating material around the suppository and by spraying the coating material on the suppository surface.

18. A method for coating of a suppository unit dosage form for rectal, vaginal or urethral administration that comprises as an active ingredient one or more fatty acids, comprising a step selected from
a) coating the inner surface of a suppository mold with coating material before molding of the suppository,
b) dipping a suppository in a liquid coating material,
c) molding or casting the coating material around a suppository, and
d) spraying the coating material on the surface of a suppository.

19. The method for coating of a suppository unit dosage form according to claim 18, wherein said suppository is as defined as in claim 1.

20. The method for coating of a suppository unit dosage form according to claim 18, wherein said coating comprises a substance selected from fatty or oleaginous bases such as cocoa butter, hard fat and hydrogenated vegetable oil, water-soluble or water-miscible bases such as polyethylene glycols, glycol-surfactant combinations and polyoxyethylene sorbitan fatty acid esters, or combinations thereof.

21. The suppository unit dosage form according to claim 1, wherein said coating comprises a biologically active agent such as local anesthetic or steroid.

22. The suppository unit dosage form according to claim 21, wherein said biologically active agent is a local anesthetic selected from one or more of lidocaine, prilocalne, procaine, benzocaine, chloroprocaine, cinchocaine, bupivacaine, tetracaine, oxetacaine, pramocaine and levobupivacaine.

23. The suppository unit dosage form according to claim 21, wherein said biologically active agent is a steroid such as hydrocortisone, prednisolone, betamethasone, fluorometholone, fluocortolone, dexamethasone, fluocinonide, fluocinolone acetonide, triamcinolone, triamcinolone acetonide and loteprednol etabonate.

24. The suppository unit dosage form according to claim 21, comprising in the coating said biologically active agent in an amount in the range of about 0.1 mg to about 20 mg.
